(19) United States
(12) Patent Application Publication
(10) Pub. No.: US 2007/0299084 A1
(43) Pub. Date: Dec. 27, 2007

(54) Indole Derivatives Useful for Treating Resistance to Antitumor Agents

(75) Inventors: Carlo Farina, Milano (IT); Paola Misiano, Gorgonzola (IT); Franco Zunino, Milano (IT)

Correspondence Address: Jay S Cinamon;ABELMAN FRAYNE AND SCHWAB, 666 Third Avenue, New York, NY, 10017-5621, US

(73) Assignees: NIKEM RESE S.R.L., Baranzate Di Bollate (IT); ISTITUTO NAZIONALE PER LO STUDIO E LA CURA DEI TUM, Milano (IT)

(21) Appl. No.: 11/579,236

(22) PCT Filed: Apr. 27, 2005
(86) PCT No.: PCT/EP05/51910

§ 371 Date: Oct. 30, 2006

(30) Foreign Application Priority Data
Apr. 30, 2004 (IT) MI 2004 A 000875

(51) Int. Cl. A61K 31/497; A61K 31/405; A61P 35/00; A61K 31/454

(52) U.S. Cl. 514/254.09; 514/323; 514/415

(57) ABSTRACT

The use of a group of indole compounds of formula (I) is described for treating tumours which have developed resistance to antitumour drugs. The compounds of formula (I) can be used in monotherapy, to treat tumours affected by drug resistance, or in co-therapy, as synergistic enhancers of the action of the aforesaid antitumour drugs. In addition, pharmaceutical compositions are described which comprise the indole derivatives of formula (I) in association with antitumour drugs the activity of which is to be enhanced.

### FIELD OF THE INVENTION

The present invention relates to the field of antitumour pharmacology. The use of indole derivatives is described for treating tumours having previously been treated with antitumour drugs, and which have developed resistant forms to said antitumour drugs.

### PRIOR ART

Systemic tumour therapy involves the use of numerous drugs belonging to different classes. Despite substantial advances in tumour cell biology knowledge and the identification of possible cell targets useful for specific therapeutic interventions, the most effective drugs in current clinical use continue to be cytotoxic agents. These drugs act by interfering with critical cell processes such as DNA functions and cell replication, possessing high cytotoxic or antiproliferative potential. For this reason an important drawback of these drugs is their toxicity and the low therapeutic index. However, the most critical limitation of conventional antitumour drugs is the phenomenon of cellular drug resistance which manifests itself in the majority of solid tumours. Indeed, with some exceptions (lymphomas, Ieukaemias, testicular tumours) in which conventional therapy results in a significant number of recoveries, human tumours in the advanced stage develop a state of resistance which is responsible for therapeutic failure. In these cases, even high dose intensive treatments and support therapies to reduce toxic effects have not produced advantageous therapeutic results. Therefore inherent resistance and acquired resistance, which manifests itself following an initial therapeutic benefit, are the principle problems of antitumour chemotherapy. In addition to the research of new molecules able to control tumour development using cytotoxic mechanisms or in a specific manner, a promising strategy for improving the effectiveness of pharmacological therapy appears to be the identification of molecules that can block the defence and survival abilities of the tumour during cytotoxic therapy treatment. Drug resistance of tumour cells is a complex and multifactorial phenomenon. Some specific changes in the tumour cell can modify the expression of a drug target (for example, DNA topoisomerase) or can increase the capacity for repairing cytotoxic damage or can reduce susceptibility to apoptosis (for example via the overerexpression of antiapoptotic factors). All these changes are directed to increase the survival ability of tumour cells. In addition the tumour cell, during the progression process, increases its defence abilities allowing it to survive and proliferate in unfavourable stressful conditions, such as the hypoxic/acid environment typical of the bulky masses of solid tumours, and to tolerate potentially lethal damage such as genotoxic damage. The expression of various defence factors (transport system, vacuolar ATPase) which play a role in reducing intracellular concentration of the drug or in its sub-cellular compartmentalization to hinder the interaction of the drug with the intracellular target, characterise a phenotype with multiple resistance which is typical of intrinsic resistance. Therefore, a pharmacological intervention aimed at these defence mechanisms, using well tolerated agents, should produce significant therapeutic advantages, improving the effectiveness of the cytotoxic drug without a substantial increase in toxicity (Oxford Textbook of Oncology, Second Edition, 2002, editors R. L. Souhami, I. Tannock, P. Hohenberger, J. C. Horiot, Oxford University Press).

The phenomenon of drug resistance (multi-drug resistance-MDR) in tumour cells is therefore characterised by the development of a resistance to drug treatment, and is the major obstacle to chemotherapy.

A large amount of clinical evidence (Hirose M., J. Med. Invest. 50, 126-135, 2003; Lin J. H., Drug Metab. Rev. 35, 417-454, 2003) shows that the MDR phenotype in tumours is associated with overexpression of proteins belonging to the ABC transporter family (P-glycoprotein or PgP, MDR, MRP; BCRP, etc.) which causes a reduction in the accumulation of a range of cytotoxic agents. The MDR phenomenon can be associated with expression changes of other proteins found on the cell membrane or within the tumour cell, such as DNA topoisomerase II (Jarvinen T. A., Breast Cancer Res. Treat. 78, 299-311, 2003), glutathione S-transferase (Townsend D. M., Oncogene 22, 7369-7375, 2003), catalase (Tome M. E., Cancer Res., 61, 2766-2773, 2001) and vacuolar ATPase (V-ATPase) (Torigoe T., J. Biol. Chem. 277, 36534-36543, 2002).

Highly effective antitumour compounds have recently been found in the so called “unusual macrolides” class (bafilomycin A1 and concanamycin) and the macrolides derived from salicylic acid (salicylhalamide, lobatamide, oximidine and apicularen). Recent data in the literature demonstrate that these products can inhibit tumour cell growth in vitro (Boyd M. R., J.P.E.T. 297, 114-120, 2001; Bowman E. J., J. Biol. Chem. 278, 44147-44152, 2003). However, research on these products is still in progress and it is difficult by the unavailability of sufficient amounts of the natural products and because of the fact that the synthesis procedures to obtain them, if available, are lengthy, very complicated and very expensive. These difficulties, together with the known intrinsic toxicity of some of the aforementioned macrolides, limit their potential use in therapy.

The patent applications WO 98/01443, WO 99/33822, WO 01/02388 and WO 01/00587 describe new indole derivatives useful for treating a series of diseases including osteoporosis, viral diseases, ulcers, autoimmune diseases, hypercholesteremic diseases, arteriosclerosis, Alzheimer's disease, angiogenic diseases, rheumatoid arthritis, diabetic retinopathy, psoriasis and tumours; the use of these products for treating resistance to antitumour agents is not described, neither is their use in combined therapy with known antitumour drugs, for the synergistic enhancement of the activity of these latter.

In the light of the known art, the need is now strongly felt for new treatments against drug resistance to antitumour agents. In particular treatments are desired which are able to sensitise tumour cells which have become resistant to classical antitumour drugs, thus making antitumour treatment more effective. In addition, new pharmaceutical compositions able to increase the activity of known antitumour agents are desired.

### SUMMARY

We have now identified a group of compounds useful for the treatment of drug resistance and of tumours associated therewith. These compounds are defined by the general formula (I)

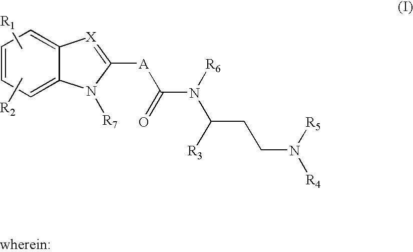

X is chosen from —CH— or —N—;

A is chosen from the groups: 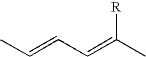
R=alkoxy, hydroxyalkoxy 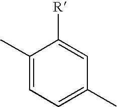
R′=hydroxy, alkoxy, hydroxyalkoxy, halogen

R1 and R2 are each independently chosen from H, halogen and alkoxy;

R3 and R4 are each independently chosen from H, alkyl, or R3 and R4, together with the atoms to which they are attached, form a 6, 7 or 8 membered heterocycle containing an atom of nitrogen, optionally substituted by one or more alkyl groups;

R5 is chosen from H, alkyl, hydroxyalkyl, alkoxyalkyl, carboxyalkyl, aminoalkyl, optionally substituted aryl or arylalkyl, optionally substituted heterocyclyl or heterocyclylalkyl, or R5 and R4, together with the nitrogen atom to which they are attached, form an optionally substituted 5-8 membered heterocyclic ring containing up to 2 heteroatoms chosen from N, O and S;

R6 and R7 are independently chosen from H and alkyl.

The aforesaid compounds of formula (I) and their preparation processes are described in the aforecited patent applications WO 98/01443, WO 99/33822, WO 01/02388 and WO 01/00587.

It has now been discovered that the compounds of formula (I) are particularly effective in inhibiting the growth of tumours which have developed resistance to known antitumour agents; in addition they are able to synergistically enhance, at lower than pharmacologically active doses, the activity of known antitumour agents such as topotecan, taxol, SN38, doxorubicin and cisplatin: the most evidence for this enhancement has been found in the case of drug resistant tumours, hence precisely under those circumstances in which the greatest need exists for enhancing the antitumour effect.

In the light of these encountered activities, the aforesaid compounds are therefore useful in both monotherapy, to treat tumours affected by drug resistance, and co-therapy as synergistic enhancers of the action of known antitumour agents; this latter treatment is effective in the case of patients already treated as well as those undergoing treatment with traditional antitumour drugs, who have developed resistant forms to said traditional antitumour drugs.

Compound of the present invention are also useful as radiosensitizers to reduce resistance to radiation therapy, a well known phemomenon occurring in many tumors.

Furthermore, the ability of compounds of the present invention to reduce in vitro chemoinvasion and in vivo metastasis, alone and in combination with known antitumor drugs, represents an excellent characteristic for a new antitumor agent, considering that metastasis is one of the major causes of death from cancer.

### DESCRIPTION OF THE FIGURES

FIG. 1: results of the co-treatment (72 hours) of HT29/Mit resistant cells with topotecan and the compound of example 2;

--▪--: Topotecan (IC₅₀>20 μM);

--●--: Topotecan+compound of example 2 at 0.5 μM (IC₅₀4 μM).

FIG. 2: results of the co-treatment (72 hours) of HT29/Mit resistant cells with topotecan and the compound of example 2 (representation according to Kern with illustration of synergism);

--▪--: Topotecan+compound of example 2 at 0.5 μM (88%);

--●--: Topotecan+compound of example 2 at 0.5 μM (104%).

FIG. 3: activity of the combination of topotecan and the compound of example 2 in HT29/Mit xenograft mice model;

--X--: controls;

--●--: Topotecan (15 mg/Kg) p.o.;

--▴--: Compound of example 2 (30 mg/kg) p.o.;

--▪--: Topotecan+compound of example 2.

### DETAILED DESCRIPTION OF THE INVENTION

In the aforesaid formula (I) all the alkyl groups, either free or contained within other substituents such as alkoxy or hydroxyalcoxy, hydroxyalkyl, etc., are generally C1-6 linear or branched alkyl groups, more preferably being C1-4 groups; even more preferably they are chosen from methyl, ethyl, n-propyl and i-propyl.

In the aforesaid halogen groups, the intended halogen atom is chosen from fluorine, chlorine, bromine or iodine, preferably being chlorine or bromine.

In the optionally substituted groups, where not indicated otherwise, the optional substituent is preferably chosen from the alkyl groups as aforedefined and keto, aryl, arylalkyl, haloaryl, hydroxyalkyl, alkoxyaryl groups.

Particularly useful meanings for the single substituents are the following:

R is preferably chosen from OMe, OEt;

R′ is preferably chosen from OMe, OEt, O-iPr, OCH₂CH₂OH, Cl and Br;

R1 and R2 are preferably chosen from H, OMe, Cl, Br;

R4 preferably forms a heterocycle, as aforedefined, with R3 or with R5 (in the aforesaid formula (I) it is intended that R4 can form a heterocycle with either R3 or R5, but not simultaneously with both).

When R4 forms a heterocycle with R3, the most preferred heterocycle is a piperidinic ring, unsubstitued or substituted with one or more substituents chosen from methyl, carboxyalkyl, hydroxyalkyl, benzyl, oxadiazolylalkyl; particularly preferred is a piperidinic ring substituted with two gem-dimethyl groups in positions adjacent to the piperidinic nitrogen.

When R4 forms a heterocycle with R5, the most preferred heterocyle is a piperazinic ring, unsubstituted or substituted in position 4 with a group chosen from methyl, phenyl, chlorophenyl, hydroxyphenyl, methoxyphenyl.

R6 and R7 are preferably chosen from H and methyl.

Specific compounds preferred for the present invention are illustrated in the following table:

| | |
|---|---|
| 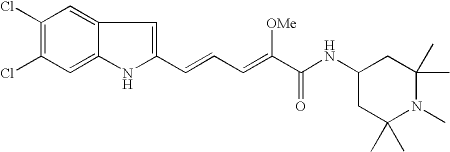 | 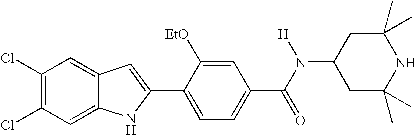 |
| 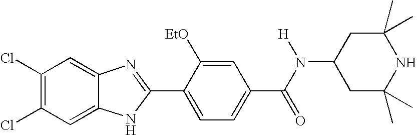 | 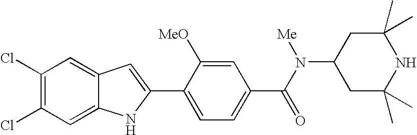 |
| 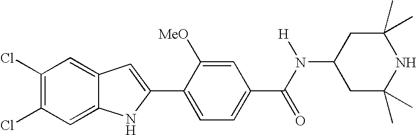 | 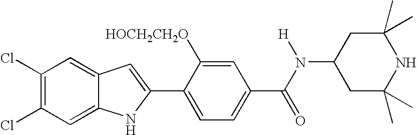 |
| 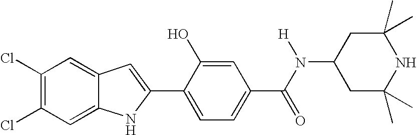 | 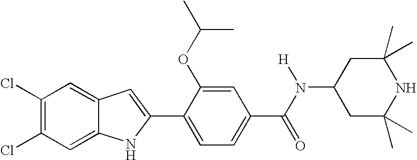 |
| 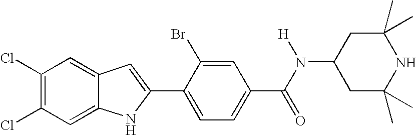 | 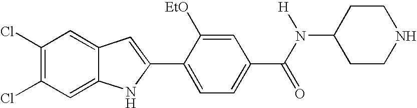 |
| 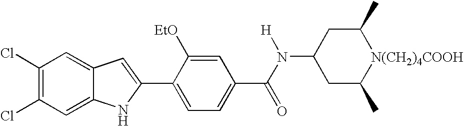 | 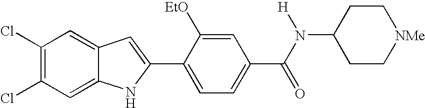 |
| 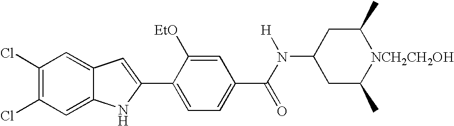 | 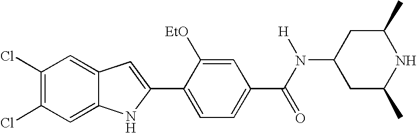 |
| 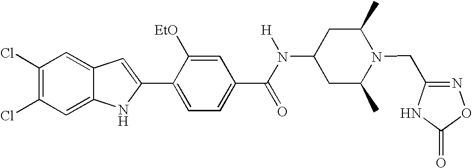 | 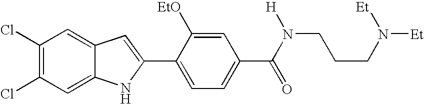 |
| 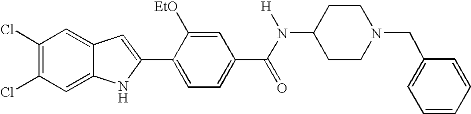 | 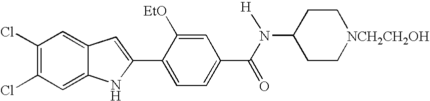 |

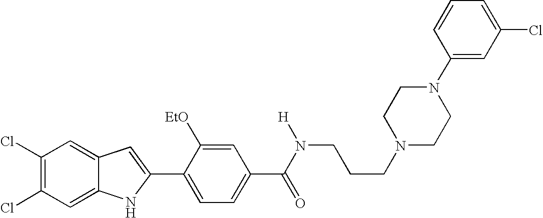

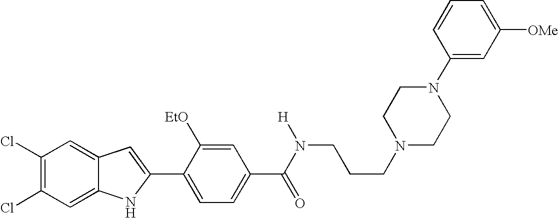

The processes for preparing the compounds of formula (I) are amply described in the cited patent applications WO 98/01443, WO 99/33822, WO 01/02388 and WO 01/00587, herein incorporated by reference.

The compounds of formula (I) can be advantageously used in the treatment of resistance to antitumour agents, thus allowing a better and more effective treatment of tumours which have lost partial or total sensitivity to said antitumour agents.

An aspect of the invention is therefore the use of one or more compounds of formula (I) in the preparation of a medicament useful for the treatment of resistance to antitumour agents. A further aspect of the invention is a method for treating resistance to antitumour agents, characterised by administering one or more compounds of formula (I) to a patient requiring them.

The treatment is aimed at to curing those tumours and that population of patients who have developed resistance following treatment with antitumour agents, this latter treatment being already concluded or still ongoing on administering the compound of formula (I).

Administration of the compound of formula (I) can be undertaken jointly with or later than administration of the antitumour agents, towards which resistance has arisen; in the case of joint administration the compound of formula (I) and the antitumour agent are preferably contained within the same pharmaceutical composition.

Examples of conventional antitumour drugs which can give rise to various manifestations of drug resistance and which can benefit from treatment combined with the compounds of formula (I) are anthracyclines (for example doxorubicin, epirubicin, mitoxantrone), camptothecins (for example topotecan, irinotecan), platinum compounds (for example cisplatin, carboplatin) and taxans (for example taxol and taxotere).

Tumours linked with the resistance phenomenon are in general those with a high level of expression in the transport systems responsible for the MDR phenomenon, such as BCRP and PgP: examples of said tumours are tumours of the digestive system such as carcinomas of the stomach, colon, liver and pancreas, tumours of the urinary system, tumours of the central nervous system such as neuroblastoma and glioma, tumour of the breast, of the bones and melanoma (Ouar Z., Biochem. J. 370, 185-193, 2003; Ohta T., J. Pathol. 185, 324-330, 1998, Nakashima S., J. Biochem.-Tokyo-134, 359-64, 2003; Altan N., J. Exp. Med. 187, 1583-1598, 1998; Martinez-Zaguilan R., Biochem. Pharmacol. 57, 1037-1046, 1999).

The compounds of formula (I) can be administered within wide dosage limits depending on the extent of the desired effect, the general condition of the patient, and the size of the tumour in question. Useful and non-limiting dosage limits are between 0.05 mg/kg and 30 mg/kg.

The administration routes are chosen according to the same considerations as aforestated and, depending on the absorbability of the active principle and its ability as a carrier, can be indifferently chosen from the intravenous, intramuscular, subcutaneous, transdermal, oral, topical, inhalation route, etc

A further aspect of the invention consists of pharmaceutical compositions in which one or more compounds of formula (I) as aforedefined are combined with one or more antitumour agents, possibly in the presence of suitable pharmaceutical excipients. These compositions exhibit an enhanced antitumour effect by virtue of the presence of the compound of formula (I).

The dosage units of these pharmaceutical compositions contain the compound of formula (I) in a quantity between 1 and 1000 mg; said units are administered so that in the patient dosages per Kg are achieved which are preferably between the aforementioned limits.

The traditional antitumour agent, present in the compositions with the compound of formula (I), is used in the normal amounts at which it is already known to be active, or in a possibly lower amount by virtue of the synergistic enhancement effect obtained by the present invention. Non-limiting reference limits for antitumour drug content, combined with the compound of formula (I) in the dosage unit, are between 0.1 and 1000 mg.

The pharmaceutical compositions of the invention can be adapted for the various administration routes as aforementioned, and can be provided for example in the form of injectable solutions, solutions for infusion, solutions for inhalation, suspensions, emulsions, syrups, elixirs, drops, suppositories, possibly coated pills, hard or soft capsules, microcapsules, granules, dispersible powders etc.

The excipients contained in these compositions are those commonly used in pharmaceutical technology, and can be used in the mode and quantity commonly known to the expert of the art.

Solid administration forms, such as pills and capsules for oral administration, are normally supplied in dosage units. They contain conventional excipients such as binders, fillers, diluents, tabletting agents, lubricants, detergents, disintegrants, colorants and wetting agents and can be coated in accordance with methods well known in the art.

The fillers include for example cellulose, mannitol, lactose and similar agents. The disintegrants include starch, polyvinylpyrrolidone and starch derivatives such as sodium starch glycolate; the lubricants include, for example, magnesium stearate; the wetting agents include for example sodium lauryl sulfate.

These solid oral compositions can be prepared with conventional mixing, filling or tabletting methods. The mixing operations can be repeated to disperse the active agent in compositions containing large quantities of fillers. These are conventional operations.

The liquid preparations can appear as such or in the form of a dry product to be reconstituted with water or with a suitable carrier at the time of use. These liquid preparations can contain conventional additives such as suspending agents, carboxymethylcellulose, aluminium stearate gel or hydrogenated edible fats, emulsifying agents, for example lecithin, sorbitan monooleate, or acacia; non aqueous carriers (which can include edible oil) for example almond oil, fractionated coconut oil, oily esters such a glycerin esters, propylene glycol or ethyl alcohol; preservatives, for example methyl or propyl p-hydroxybenzoate or sorbic acid and if desired, conventional flavours or colorants.

The oral formulations also include extended release conventional formulations, such as enteric coated pills or granules.

For parenteral administration, fluid dosage units can be prepared, which contain the compound and a sterile carrier. The compound, depending on the carrier and concentration, can be suspended or dissolved. The parenteral solutions are normally prepared by dissolving the compound in a carrier and sterilizing by filtration, before filling suitable vials or ampoules and sealing. Adjuvants such as local anaesthetics, preservatives and buffering agents can be advantageously dissolved in the carrier. In order to increase stability, the composition can be frozen after filling the vial and the water removed under vacuum. The parenteral suspensions are prepared essentially in the same way, with the difference that the compound can be suspended rather than dissolved in the carrier, and can be sterilized by exposure to ethylene oxide prior to being suspended in the sterile carrier. A surfactant or humectant can be advantageously included in the composition to facilitate uniform distribution of the compound of the invention.

As is the common practise, the compositions are normally accompanied by written or printed instructions, for use in the treatment concerned.

The following experimental methods illustrate the in vitro and in vivo activities of the compounds of the invention, without limiting the scope thereof.

Experimental Part

### In Vitro Studies

1. Biochemistry

1.1 Determination of Vacuolar ATPase Inhibition in Human Osteoclastoma (hOc)

Osteoclast-like giant cells isolated from human osteoclastoma are homogenized using a glass-teflon homogeniser (1000 rpm) and the material is centrifuged for 20 minutes at 6000 g. The resultant pellet is resuspended and centrifuged at 100000 g for 60 minutes to sediment the microsomal fraction. The resultant pellet is resuspended in medium at pH 7.4 and stored under liquid nitrogen.

Inhibition of bafilomycin sensitive ATPase activity is assayed by measuring the release of inorganic phosphate during 30 minutes of incubation, at 37° C., of the human osteoclastoma microsomal fraction in 96-well plates. The reaction medium contains 1 mM ATP, 10 mM Hepes-Tris buffer pH 8, 50 mM KCl, 5 μM valinomycin, 5 μM nigericin, 1 mM CDTA-Tris, 100 μM ammonium molybdate, 0.2 M sucrose and the microsomal fraction (20 μmg protein/ml). The reaction is initiated by adding MgSO₄ and terminated, after 30 minutes, by adding 4 volumes of the reagent malachite green, prepared according to Chan K., Anal. Biochem. 157, 375-380, 1986.

1.2 Determination of Vacuolar ATPase Inhibition in Bovine Chromaffin Cells Membranes (BCG)

About 20 adrenal glands (Cidon S., J. Biol. Chem. 258, 2892-2898, 1983) are removed from healthy bovines; the medulla is quickly separated from the cortex, which is discarded. The medulla is homogenised at 4° C. with a suitable medium at pH 7.5, then filtered. The remaining solid material is further homogenised, filtered and recombined with the preceding filtrate, resuspended and centrifuged at 1000 g for 15 minutes; the supernatant obtained is centrifuged at 10000 g for 20 minutes. The resultant pellet is resuspended and stratified through a sucrose gradient formed from a lower part of 15 ml 1.5 M sucrose and an upper part of 10 ml 1.2 M sucrose. After overnight centrifugation at 4° C. with a SW28 rotor at 20000 rpm the chromaffin cells sediment into a pellet. This latter is resuspended, centrifuged at 3000 g for 10 minutes, and the supernatant obtained is centrifuged at 200000 g for 60 minutes. The pellet is then resuspended in 4 ml of a suitable medium containing 0.2 μg/ml pepstatin A and 0.4 μg/ml leupeptin, and stored under liquid nitrogen.

The method for ATPase inhibition assay is the same as that followed for the osteoclastoma.

2. Cell Pharmacology

2.1 Cell Lines and Culture Conditions

Human Colon Carcinoma:

HT29 and HT29/Mit (line obtained by prolonged exposure to mitoxantrone, and characterised by overerexpression of BCRP, which confers cross resistance to topotecan, irinotecan and to its metabolite SN38): maintained in McCoy 5A medium+10% FCS.

LoVo and LoVo/Dx (line obtained by prolonged exposure to doxorubicin and characterised by overerexpression of P-glycoprotein, which confers resistance to doxorubicin): maintained in HAM-F12 medium+10% FCS.

HCT116 maintained in RPMI 1640 medium+10% FCS.

Human Neuroblastoma:

SH-SY5Y and SK-N-BE(2): maintained in HAM-F12 medium+10% FCS.

Human Hepatic Carcinoma:

HepG2: maintained in EMEM medium+10% FCS.

Human Ovarian Carcinoma:

A2780: maintained in RPMI 1640 medium+10% FCS.

Human Lung Carcinoma:H460: maintained in RPMI1640 medium+10% FCS.

2.2 Scheme of the Antiproliferative Activity Experiment (Treatment Time: 72 Hours)

The cells (HT29 and HT29/Mit:. 40,000 cells/ml, LoVo, LoVo/Dx and HCT116: 50,000 cells/ml) are seeded in 100 μl of the respective culture media in 96-well plates. 24 hours after seeding, an aliquot (10 μl) of drug at the various concentrations is added. In the samples in which the effect of the combination of two compounds is to be tested the inhibitor is added immediately before the cytotoxic. For each dose or combination of doses/drugs the effect of the treatment is determined in 4-8 replicates.

After 72 hours of treatment the antiproliferative effect is evaluated using the sulforhodamine B (SRB) assay: the cells are fixed by adding 25 μl of 50% TCA to each well and left for 1 hour at 4° C. After washing them with water and allowing them to dry, 100 μl of 0.4% SRB in 1% acetic acid are added and left for 30 minutes at room temperature. After 4 washes in 1% acetic acid, they are left to dry, then the dye fixed by the proteins is dissolved under basic conditions with 100 μl 10 mM cold Tris and the solution is read using a spectrophotometer at 550 nm.

Data Analysis

Percentage cell growth is calculated as the optical density of treated samples compared to the optical density of controls (untreated cells).

The Combination Index (C.I.) was determined according to Kern's method, 1988 (who continued from Drewinko B, 1976) (Kern D. H., Cancer Res. 48, 117-121, 1988; Drewinko B., Cancer Biochem. Biophys. 1, 187-195, 1976) by means of the following formula: (Sfa+SFb)/Sfab, where SFa and SFb are the fractions of cells surviving to treatment with compound a and b, respectively; SFab is the fraction surviving to the combination of compounds a and b. If the result is=1, the interaction between the two compounds is additive; if it is>1, the interaction between the two compounds is synergistic; if it is <1, the interaction between the two compounds is antagonistic.

2.3 Scheme of the Antiproliferative Activity Experiment (Treatment Time: 48 Hours)

The cells (concentration: 30,000 cells/ml) are seeded in 90 μl of the respective culture media in 96-well plates. 24 hours after seeding, an aliquot (10 μl) of the drug at the various concentrations is added (for each concentration there are 3 replicates). After 48 hours of treatment the antiproliferative effect is evaluated with a luminescence assay (Perkin Elmer Life Sciences ATPlite):

50 μl of a lysis solution are added to each well followed by an equal volume of a solution containing luciferase and D-luciferin. The ATP present in all the metabolically active cells brings about the transformation reaction of D-luciferin, catalysed by luciferase, to produce a luminescent signal as described in the following scheme:
ATP+D-luciferin+O₂→Oxyluciferin+AMP+PPᵢ+CO₂+Light

The luminescence produced (expressed in counts per second, CPS) is measured by means of a microplate scintillation analyzer (Perkin Elmer Life Sciences Top Count).

Data Analysis

Percentage inhibition of luminescence in the treated cells compared to the control is calculated; concentration-response curves are then analysed using Grafit v.5.0.

2.4 Scheme of Apoptosis Experiment

1.5×10⁶ cells/sample are seeded. After 24 hours the cells are treated with the compounds for 48 or 72 hours. At the end of the treatment, the cells are detached with Trypsin/EDTA, washed in PBS (phosphate buffered solution) and incubated for 45 minutes at room temperature in 1 ml of 4% paraformaldehyde.

The cells are then washed with PBS and resuspended in 100 μl of permeabilizing solution (0.1% triton in 0.1% sodium citrate) for 2 minutes in ice. After a further wash, the cells are resuspended in 50 μl of Tunel reaction mix (Boehringer Mannheim) and left at 37° C. for 1 hour in the dark. After washing in PBS, the cells are resuspended in PBS and analysed by cytofluorimeter or examined by fluorescence microscope.

2.5 Scheme of Irradiation Experiment

HT29 cells (50,000 cells/ml) were seeded and 24 h later they were irradiated with a 137 Cs source delivering 0.13 Gy/s, in presence and in absence with the test compound. After 72 h treatment, adherent cells were collected, washed in PBS and counted to evaluate the cytotoxic effect of the treatment.

2.6 Scheme of Migration and Invasion Assays

H460 cells were seeded in complete medium and treated with different compound concentrations for 24 h. Then, cells were harvested and transferred to 24-well Transwell chambers (Costar) in serum-free medium in the following ways:

migration assay: 1.2×10⁵ cells/well were seeded in the upper chamber, and the drug was added, in the same concentrations utilized before, in both upper and lower chambers. After 4 h of incubation at 37° C., migrated cells were fixed in 95% ethanol, stained with a 2% crystal violet in 70% ethanol solution, and counted by an inverted microscope.

invasion assay: Transwell membranes were coated with 12.5 μg/well of Matrigel (BD Biosciences) and dried for 24 h. After this, 2.4×10⁵ cells/well were seeded onto the artificial basement membrane in upper chamber, and drug was added as described for migration assay. After 24 h of incubation at 37° C., cells that invaded the Matrigel and migrated to the lower chamber were stained and counted as described for migration assay.

2.7 Results

Antiproliferative Effect on Tumour Cells (Single Treatment)

The results are given in Table 1.

| | | v-ATPase | | | | | | | | | |
|---|---|---|---|---|---|---|---|---|---|---|---|
| | | Activity | Antiproliferative activity | | | | | | | | |
| | | (IC₅₀, μM) | (IC₅₀ ± S.D., μM) | | | | | | | | |
| Compound | Structure | hOc | HT-29 | HT-29/Mit | LoVo | LoVo/Dx | HCT116 | HepG2 | SHSY-5Y | SK-N-BE(2) | A2780 |
| | | | 72 h. | | | | | 48 h. | | | |
| | | | treatment | | | | | treatment | | | |
| Cisplatin | | | 6.8 | | 0.7 | 0.8 | 5.4 | >25 | 7.3 ± 3.3 | 10.1 ± 3.5 | 8.1 ± 2.1 |
| Topotecan | | | 0.07 ± 0.01 | 50.6 | | | | | | | |
| Bafilomycin | | 0.0001 | 0.003 ± 0.002 | 0.005 | | | | <0.006 | | | |
| A1 | | | | | | | | | | | |
| Example 1 | 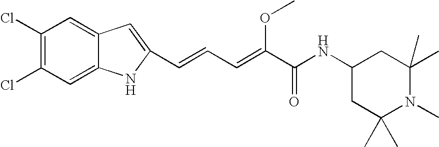 | 0.029 | 3.3 ± 0.4 | 1.0 | | | | 1.1 ± 0.1 | | | |
| Example 2 | 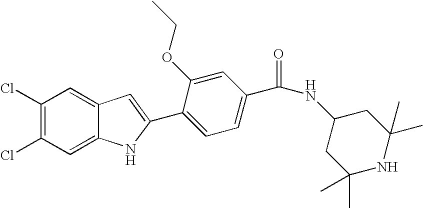 | 0.025 | 1.0 ± 0.1 | 0.9 ± 0.2 | 2.4 ± 1.4 | 3.0 ± 1.3 | 2.4 ± 0.9 | 2.0 ± 0.1 | 6.8 ± 0.5 | 3.7 ± 0.2 | 1.9 ± 0.1 |
| Example 3 | 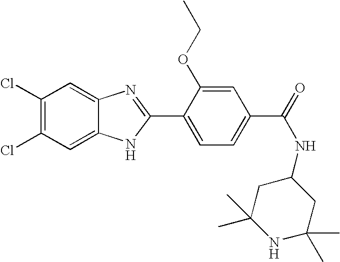 | 0.008 | 0.56 ± 0.26 | 0.27 | | | | 0.71 ± 0.01 | | | |
| Example 4 | 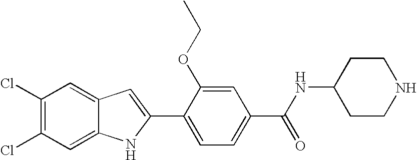 | 0.370 | 4.7 | 3.6 | | | | | | | |
| Example 5 | 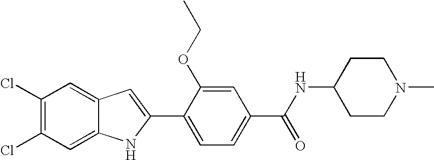 | | 3.8 | 2.3 | | | | | | | |
| Example 6 | 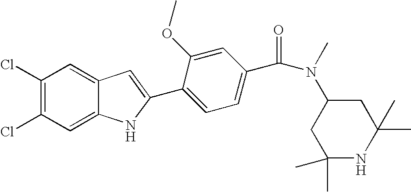 | >3 μM | 4.7 | 3.3 | | | | 0.92 ± 0.01 | | | |

Table 1 shows that the compounds of examples 1-6 of the present invention demonstrate an antiproliferative activity in the human tumour cell lines used, whether after 72 hours treatment (HT29, HT29/Mit, LoVo, LoVo/DX and HCT116) or 48 hours treatment (HepG2, SHSY-5Y, SK-N-BE(2) and A2780). In particular, the compound of example 2 shows a high antiproliferative potency (very comparable to known antitumour agents) in all tumour cell lines. This antiproliferative activity is maintained in variants of human colon carcinoma lines made resistant to cytotoxic agents of clinical interest associated with the MDR phenomenon, among which the HT29/Mit line (obtained by prolonged exposure to mitoxantrone and characterised by BCRP overexpression, which confers cross-resistance to topotecan, to irinotecan and to its metabolite SN38) and the LoVo/Dx line (obtained by prolonged exposure to doxorubicin and characterised by overexpression of P-glycoprotein ).

Antiproliferative Effect on Tumour Cells (Combined Treatment With Known Antitumour Agents)

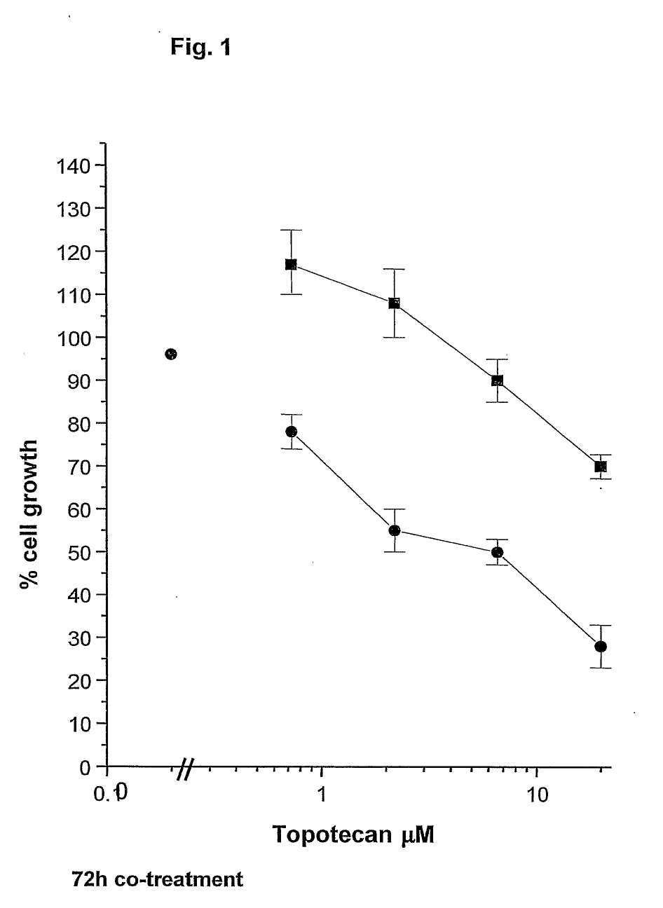

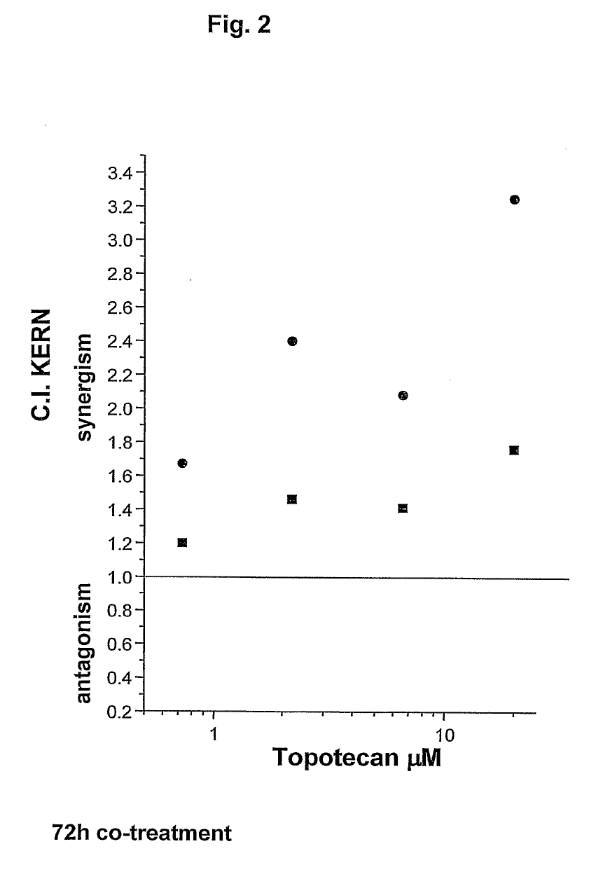

In the HT29 and HT29/Mit models, the compound of example 2 of the invention has produced an enhanced antiproliferative activity of topotecan at subtoxic and subactive concentrations (<1 μM). The synergism between the compound of example 2 of the invention and topotecan is illustrated in FIGS. 1 and 2. The experiment was performed on the HT29/Mit line (resistant to mitoxantrone and topotecan): by co-treating these cells for 72 hours with topotecan and with the compound of example 2 at a concentration of 0.5 μM (which does not itself inhibit cell growth), a considerable enhancement of topotecan activity is found (the IC₅₀ for topotecan goes from >20 μM to 4 μM).

As illustrated in FIG. 2, in which the Combination Index according to Kern has been calculated (see in vitro studies 2.2), there is a clear synergistic effect with the combination of topotecan and the compound of example 2 at 0.5 μM. In this graph a further experiment on the combination of topotecan and the compound of example 2 at 0.5 μM has been added. In another tumour cell line, the HCT116 line, the compound of example 2 has demonstrated a significant synergistic effect with cisplatin at subtoxic and subactive concentrations. In addition, in the LoVo line a synergism was observed between the compound of example 2 and both taxol and SN38, the active metabolite of irinotecan. The synergistic interaction with SN38 is even more evident in the LoVo/DX variant which displays the MDR phenotype. Synergistic interactions were also observed between the compound of example 2 and doxorubicin or taxol.

The compound of example 2 also induces a high level of apoptosis in HT29 line.

Inhibitory Effect on Tumoral Cells Migration and Invasion:

Compound of example 2, after 24 h incubation, greatly inhibited the H460 chemotaxis in a concentration-related manner: the inhibition was 51% at 2.2 μM, and 92% at 5.7 μM. Furthermore, compound of example 2 very potently inhibited the H460 chemoinvasion, evaluated by Matrigel assay, with 92% and 100% inhibition at 2.2 and 5.7 μM respectively.

### In Vivo Studies

1.1 Model of HT29/Mit Human Colon Carcinoma Xenografts-Antitumor Activity

Female athymic Swiss nude mice (8-10 weeks old) (Charles River, Calco, Italy) were used for the experiments. The animals were maintained at constant temperature and humidity, and were allowed to eat and drink freely. The experimental protocol was approved by the Ethics Committee for Animal Experimentation of the Istituto Nazionale Tumori of Milan.

The antitumour effectiveness of the compounds of the invention under discussion was tested on athymic mouse models implanted with HT29 and/or HT29/Mit tumour cells: this latter variety is highly resistant to topotecan treatment. The tumour cells were implanted in vivo via subcutaneous injection of 10⁷ cells taken from in vitro cultures. Randomized groups of five mice with bilateral subcutaneous tumours were used for the experiment.

Topotecan or other known antitumour agents (dissolved in distilled water or an appropriate solvent) and the compounds of the invention (dissolved in Cremophor EL:ethanol:saline solution in the proportions 5:5:90, or in an appropriate solvent) were administered orally from the third day, alone or in combination, in agreement with a treatment scheme selected in an appropriate manner depending on the type of compound to be studied.

The weight (or volume) of the tumour in treated mice compared to controls is represented graphically on the y-axis against time (x-axis).

1.2 Model of H460 Human Non-Small Cell Lung Carcinoma Xenograft-Antitumor and Antimetastatic Activities

Female athymic Swiss nude mice (8-10 weeks old) (Charles River, Calco, Italy) were used for the experiments, as described above.

H460 cells were injected i.p. into nude mice, adapted to grow as ascitis and maintained in vivo by i.p. passages (5×10⁶ cells/mouse in 0.5 ml PBS) (Pratesi G., Br. J. Cancer 63, 71-74, 1991). Briefly, cells were collected from the donor mice about 7 days after inoculum. After washing, cell number and viability were determined by trypan blue exclusion. Such procedure allowed to obtain a single cell suspension easily available for s.c or i.v. injection.

The effects of the compounds of the invention and/or topotecan on the growth of primary tumors and spontaneous lung metastasis were tested in mice inoculated s.c. in the right flank with H460 ascitic tumor cells (2×10⁶/mouse). Each control or drug-treated group included 9-11 mice. The s.c. tumor growth was followed by biweekly measurements of tumor diameters with a Vernier caliper. Drug treatment was delivered orally, for 8 weeks, from day 1. Topotecan was delivered at the dose of 1 mg/kg and compounds of the invention were delivered at the dose of 30 mg/kg; in the combination group the compounds of the invention were delivered almost 1 hour after topotecan treatment. Control mice were solvent-treated orally in parallel with drug treatments.

Drug efficacy was assessed as mean percentage tumor weight inhibition in drug-treated versus control mice expressed as tumor weight inhibition % (TWI %)=100−(mean tumor weight treated/mean tumor weight control×100), evaluated during and after drug treatment. Drug tolerability was assessed in tumor-bearing mice as either lethal toxicity, i.e., any death in treated mice occurring before any control death, or percentage body weight loss (BWL %)=100−(body weight on day x/body weight on day 1×100), where x represents a day after or during the treatment period.

At day 63, tumor-bearing mice were sacrificed by cervical dislocation and their lungs were removed and weighed. Lung lobes were spliced between two glass slides and the metastatic nodules were macroscopically counted against a bright light (Corti C, J. Cancer Res. Clin. Oncol. 122, 154-60, 1996). Spontaneous lung metastases were present in 100% of control mice. Reading of metastasis was performed by two independent observers, unaware of the experimental group, with an interobserver reproducibility >95%. The metastatic nature of these areas was confirmed by histological analysis of digital images obtained by Image Analysis System software (Delta System, Rome, Italy).

1.3 Results

Antitumor Activity (HT29/Mit Xenograft Model)

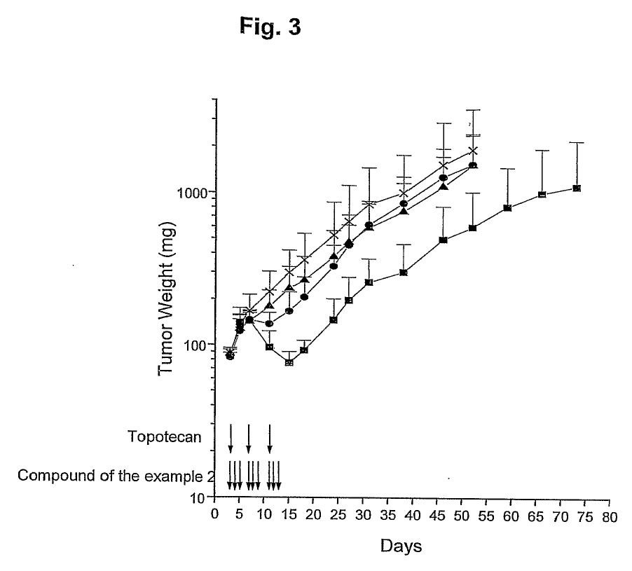

The results of the in vivo experiment are given in FIG. 3.

From the data in FIG. 3, it can be seen that in the HT29/Mit xenograft model oral treatment with topotecan every 4 days for 3 administrations causes a low inhibition of tumour growth (about 30-40%). The compound of example 2 is also poorly effective per se in controlling tumour growth (treatment of 30 mg/kg p.o.). However, the administration of the compound of example 2 and topotecan combined causes substantial statistically significant tumour growth inhibition (about 75%) which appears to persist even after drug treatment has been suspended.

Antitumor and Antimetastatic Activities (H460 Xenograft Model)

The results of the in vivo experiment are given in Table 2.

Both topotecan (1 mg/kg p.o.) and compound of example 2 (30 mg/kg p.o.) were able to display a clear, statistically significant antitumoral activity (57 and 58% tumor growth inhibition respectively) on the H460 primary tumor. The combination of both drugs reached a statistically significant 74% growth tumor inhibition at day 56 of treatment. Very interestingly, the number of lung metastasis observed was reduced in both topotecan and compound of example 2-treated groups. The combination of both drugs produced a highly significative inhibition (81 %) of number of metastasis. No toxicity was observed in treated animals.

TABLE 2

| | | | | Lung | | | |
|---|---|---|---|---|---|---|---|
| | Dose | Max^{a} TWI | | Weight (mg) | No. Metastasis | | |
| Drug | mg/kg | (day 56) | LCK^{b} | (mean ± S.D.) | (range) | Inhibition % | Tox/Tot^{c} |
| Controls | — | — | — | 191 ± 44 | 26 (5-80) | — | 0/12 |
| Topotecan | 1 | 57* | 0.5 | 169 ± 16 | 11 (4-23) | 58 | 0/10 |
| Compound | 30 | 58* | 0.6 | 177 ± 30 | 15 (3-56) | 42 | 0/10 |
| of example 2 | | | | | | | |
| Topotecan + | 1 | 74** | 1.3 | 165 ± 20 | 5 (1-9) | 81^{‡,†,°} | 0/10 |
| compound | 30 | | | | | | |
| of example 2 | | | | | | | |

*P < 0.01 and
**P < 0.001 vs controls, by Student t'test.
‡P < 0.001 vs controls,
†P < 0.05 vs topotecan-treated mice,
°P < 0.005 vs compound of example 2-treated mice; by Mann-Whitney U test.
^{a}Maximal TWI (Tumor weight inhibition) achieved
^{b}LCK = Log₁₀ Cell Kill
^{c}Any death in treated mice occurring before any control mice

1. Method for treating resistance to antitumour agents, comprising the administration of a compound of formula (I) 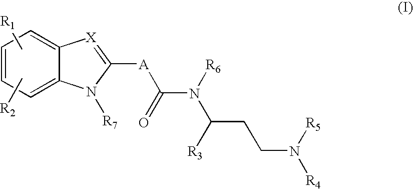
wherein:
X is chosen from —CH— or —N—;
A is chosen from the groups: 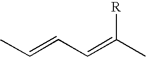
R=alkoxy, hydroxyalkoxy 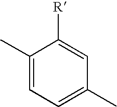
R′=hydroxy, alkoxy, hydroxyalkoxy, halogen
R1 and R2, are each independently chosen from H, halogen and alkoxy;
R3 and R4 are each independently chosen from H, alkyl, or R3 and R4, together with the atoms to which they are attached, form a 6, 7 or 8 membered heterocycle containing an atom of nitrogen, optionally substituted by one or more alkyl groups;
R5 is chosen from H, alkyl, hydroxyalkyl, alkoxyalkyl, carboxyalkyl, aminoalkyl, optionally substituted aryl or arylalkyl, optionally substituted heterocyclyl or heterocyclylalkyl, or R5 and R4, together with the nitrogen atom to which they are attached, form an optionally substituted 5-8 membered heterocyclic ring containing up to 2 heteroatoms chosen from N, O and S;
R6 and R7 are independently chosen from H and alkyl, to a patient in need thereof.
[in the preparation of a medicament useful for the treatment of resistance to antitumour agents.]

2. Method as claimed in claim 1, wherein R is chosen from OMe, OEt;

3. Method as claimed in claim 1, wherein R′ is chosen from OH, OMe, OEt, OiPr, OCH₂CH₂OH, Cl, Br.

4. Method as claimed in claim 1, wherein R1 and R2 are chosen from Cl, Br, OMe, H.

5. Method as claimed in claim 1, wherein R3 and R4 together form a piperidinic ring, optionally substituted with one or more groups chosen from methyl, gem-dimethyl, carboxyalkyl, hydroxyalkyl, benzyl, oxadiazolylalkyl.

6. Method as claimed in claim 1, wherein R4 and R5 together form a piperazinic ring optionally substituted with a group chosen from Me or phenyl optionally substituted by Cl, OH or OMe.

7. Method as claimed in claim 1, wherein the compound of formula (I) is chosen from the following compounds: (2Z,4E) N-(1,2,2,6,6-pentamethyl-piperidin-4-yl)5-(5,6-dichloro-1H-indol-2-yl)-2-methoxy-penta-2,4-dienamide, 4-(5,6-dichloro-1H-indol-2-yl)-3-ethoxy-N-(2,2,6,6-tetramethyl-piperidin-4-yl)-benzamide, 4-(5,6-dichloro-1H-benzimidazol-2yl)-3-ethoxy-N-(2,2,6,6-tetramethyl-piperidin-4yl)-benzamide, 4-(5,6-dichloro-1H-indol-2-yl)-3-ethoxy-N-piperidin-4-yl-benzamide, 4-(5,6-dichloro-1H-indol-2-yl)-3-ethoxy-N-(1-methyl-piperidin-4-yl)-benzamide, 4-(5,6-dichloro-1H-indol-2-yl)-3-methoxy-N-methyl-N-(2,2,6,6-tetramethyl-piperidin-4-yl)-benzamide.

8. Method as claimed in claim 1, wherein the patient is affected from a [wherein the tumour which has developed resistance is a] tumour of the digestive system, urinary system, central nervous system, breast, bones or a melanoma.

9. Method as claimed in claim 1, wherein the patient is already treated or is undergoing treatment with one or more antitumour drugs.

10. Method as claimed in claim 9, wherein the compound of formula (I) enhances the activity of said antitumour drugs.

11. Method as claimed in claim 10, wherein the compound of formula (I) is effective as antimetastatic, or radiosensitizer in the radiation therapy.

12. Method as claimed in claim 10, wherein said antitumour drugs are chosen from anthracyclines, camptothecins, platinum compounds and taxans.

13. Pharmaceutical composition comprising a compound of formula (I) as claimed in claim 1, in association with an antitumour drug.

14. Pharmaceutical composition as claimed in claim 13, wherein said antitumour drug is chosen from anthracyclines, camptothecins, platinum compounds and taxans.

15. Pharmaceutical composition as claimed in claim 13, in the form of an injectable solution, a solution for infusion, a solution for inhalation, a suspension, an emulsion, a syrup, an elixir, drops, a suppository, a coated or uncoated pill, a hard or soft capsule, a microcapsule, granules or a dispersible powder.

16. Pharmaceutical composition as claimed in claim 13, in the form of a dosage unit comprising the compound of formula (I) in a quantity between 1 and 1000 mg.

17. Pharmaceutical compositions as claimed in claim 13, in the form of a dosage unit comprising the antitumour drug in a quantity between 0.1 and 1000 mg.
